(19) United States
(12) Patent Application Publication
(10) Pub. No.: US 2012/0253151 A1
(43) Pub. Date: Oct. 4, 2012

(54) Multiple Wavelength Pulse Oximetry With Sensor Redundancy

(75) Inventors: Daniel Lisogurski, Boulder, CO (US); Bo Chen, Louisville, CO (US)

(73) Assignee: Nellcor Puritan Bennett LLC, Boulder, CO (US)

(21) Appl. No.: 13/076,232

(22) Filed: Mar. 30, 2011

(51) Int. Cl. A61B 5/1455

(52) U.S. Cl. 600/323

(57) ABSTRACT

Systems and method are provided that enable a spectrophotometric system to obtain reasonably reliable measurements even in situations when some of the emitters included in a sensor system have become inoperable. In certain embodiments, the spectrophotometric system may include two or more light emitters. The light emitters may be used to derive measurements suitable for pulse oximetry, hemometry, and/or aquametry. The failure of one or more of the emitters may still allow for the derivation of certain measurements by using the emitters that remain in an operational state.

### BACKGROUND

This section is intended to introduce the reader to various aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

The present disclosure relates generally to medical devices and associated sensors used for sensing physiological parameters of a patient. In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

Certain techniques for monitoring desired physiological characteristics of a patient are generally referred to as spectroscopy, and the devices built based upon such techniques include spectrophotometric devices such as pulse oximeters. Spectrophotometric devices may be used to measure various physiological characteristics, including blood-oxygen saturation of hemoglobin (SpO₂) in arterial blood, concentrations of hemoglobin (i.e., hemometry), water concentration measurements (i.e., aquametry), volume of individual blood pulsations supplying the tissue, and/or the rate of blood pulsations corresponding to each heartbeat of a patient.

Spectrophotometric devices typically utilize a non-invasive sensor suitable for emitting light into a patient's tissue and capable of photoelectrically detecting the absorption and/or scattering of the emitted light in such tissue. One or more of the above physiological characteristics may then be calculated based upon the amount of light absorbed and/or scattered. More specifically, the light passed through the tissue is typically selected to be of one or more wavelengths that may be absorbed and/or scattered by constituents of interest in an amount correlative to the amount of the constituent present in the blood. The amount of light absorbed and/or scattered may then be used to estimate, for example, the amount of constituent in the tissue using various algorithms. This determination may be performed in a monitor coupled to the sensor that receives the necessary data for the constituent calculation. Measurement techniques that rely upon measuring the changing absorption or scattering of light due to the changing volume of a body organ are known as photoplethysmography. For example, pulse oximetry may rely upon the changing volume of blood vessels due to the pulsation of blood.

Spectrophotometric devices may include two or more light emitters optimized for use in measuring a variety of physiological parameters. For example, one emitter may be configured to emit light at certain wavelengths, while a second emitter may be configured to emit light a different wavelengths. Unfortunately, one (or more) of the light emitters may become inoperative, and inaccuracies may result from using less than the full complement of light emitters.

### SUMMARY

Certain aspects commensurate in scope with the disclosed embodiments are set forth below. It should be understood that these aspects are presented merely to provide the reader with a brief summary of certain embodiments and that these aspects are not intended to limit the scope of the disclosure. Indeed, the disclosure and/or claims may encompass a variety of aspects that may not be set forth below. In particular, the embodiments below illustrate examples from pulse oximetry; however, one of skill in the art will appreciate that such techniques are equally applicable to other photoplethysmography measurements.

The present techniques may provide for reasonably reliable estimates of physiological parameters, such as arterial oxygen saturation, by using a recovery or limp mode of operation should one or more light emitters of the spectrophotometric system fail or become otherwise inoperative. The techniques may be applicable to reflectance and transmission pulse oximetry, hemometry (measurement of a quality of blood such as total hemoglobin), aquametry (measurement of body water), and other spectroscopic systems that may include multiple light emitting devices. As one example, the disclosed techniques may be useful for pulse oximetry systems that include three or more light emitters (e.g., LEDs). Pulse oximetry systems may be used to measure, for example, arterial oxygen saturation (SpO₂), pulse rates, volumes of individual blood pulsations supplying the observed tissue, and so forth. In certain pulse oximetry systems, three or more LEDs may be used to more accurately estimate the arterial oxygen saturation in patients having low or high saturation levels. In these pulse oximetry systems, for example, one LED may be configured to emit light at wavelengths near 660 nm, a second LED may be configured to emit light at wavelengths near 730 nm, and a third LED may be configured to emit light at wavelengths near 900 nm. The use of three or more LEDs may increase the accuracy of the SpO₂ measurements by selecting two LEDs pairs capable of higher accuracy under certain conditions. For example, the 730 nm-900 nm emitter pair may be selected to more accurately calculate SpO₂ at lower arterial oxygen saturation levels (e.g., below 75%), and the 600 mm-900 nm emitter pair may be selected to more accurately calculate SpO₂ at higher arterial oxygen saturation levels (e.g., above 85%). All three emitters may be used to calculate SpO₂ in a transition region between low and high arterial oxygen saturation levels (e.g., between 75% and 85%).

Should one or more of the emitters fail or become otherwise inoperative, the pulse oximetry system may enter a limp mode of operation capable of providing reasonable measurements. That is, the emitters that remain operational may be used to derive measurements having an accuracy suitable for clinical use. In one example, different calibration coefficient tables may be used to derive useful measurements if an emitter fails, as compared to the use of more focused calibration coefficient tables used when all of the emitters are operating normally. For example, should the 660 nm emitter fail, the 730 mm-900 nm emitter pair may be used with broader calibration curves that include calibration coefficients useful in deriving a wider range of SpO₂ values may be used, including calibration coefficients for use with low and high levels arterial oxygen saturation levels. Likewise, should the 730 nm emitter fail, the 660 nm-900 nm emitter pair may be used with broader calibration curves suitable for deriving SpO₂ values at wider arterial oxygen saturation levels, including low and high levels. The pulse oximetry system may also notify the user or clinician of the status of the sensor, including the failure of any of the emitters and corresponding changes in the accuracy of measurements. Such codes or alarms may then be communicated through visual text and/or audible alarms, thus alerting a user or a clinician of the operational status of the sensor 12.

It should be noted that a particular sensor may be used to make multiple types of measurements. For example, a pulse oximetry sensor may be used to measure SpO₂ and pulse rate. While the measurement of SpO₂ customarily requires at least two emitters, pulse rate measurements may be made with a single emitter. In the event of an emitter failure, the system may discontinue certain measurements while continuing others. For example, a pulse oximetry system that stops reporting SpO₂ due to emitter failure may continue to report pulse rate. Such a system may also warn the user if the preferred emitters have failed. For example, it may be preferable to calculate pulse rate using a 900 inn emitter. If this emitter fails, the system may continue to report pulse rate based on another emitter, with a suitable warning of reduced accuracy or reliability.

### BRIEF DESCRIPTION OF THE DRAWINGS

Advantages of the disclosed techniques may become apparent upon reading the following detailed description and upon reference to the drawings in which:

FIG. 1 depicts a diagram of an embodiment of a spectrophotometric system;

FIG. 2 depicts an embodiment of a sensor having multiple LEDs with a photodetector positioned to receive the LED signals in a transmission mode of operation;

FIG. 3 depicts an embodiment of a sensor having multiple LEDs with a photodetector positioned to receive the LED signals in a reflectance mode of operation;

FIG. 4 illustrates an embodiment of a flow diagram of a method of recovering from the failure of one or more light emitters;

FIG. 5 is a block diagram of an embodiment of a spectrophotometric system; and

FIG. 6 is a diagram of an embodiment of a light detector signal useful in deriving the operational status of an emitter.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In one embodiment, a sensor may be configured to permit the use of two light emitter pairs, one emitter pair set to emit light at near 660 nm and 900 nm and a second emitter pair set to emit light at near 730 nm and 900 nm, where the same 900 nm LED may be used in each pair. For example, light from the 730 nm-900 nm emitter pair may then be used with calibration curves or tables suitable for accurately calculating SpO₂ when the arterial oxygen saturation is low (e.g., below 75%). Likewise, light from the 660 nm-900 nm emitter pair may be used with calibration curves or tables suitable for accurately calculating SpO₂ when the arterial oxygen saturation is high (e.g., greater than 84%). Light from both emitter pairs may be used with calibration curves or tables suitable for accurately calculating SpO₂ in a transition region, e.g., the region between the low and high oxygen saturation. Various techniques may be used to more accurately compute the SpO₂ at the various levels, such as the techniques disclosed in patent application Ser. No. 12/888,226 to Bo Chen et al., filed on Oct. 22, 2010, and entitled “Wavelength Switching for Pulse Oximetry,” which is hereby incorporated by reference for all purposes as if fully set forth herein. Accordingly, more precise measurements may be made in a variety of patients, such as neonatal patients, adults, and cardiac patients.

The techniques disclosed herein enable the sensor to continue providing useable information even in the event of the failure of certain emitters. In one example, failure of one of the emitters may be detected, and appropriate action may then be taken based on the current SpO₂ level. In this example, if the 660 nm emitter fails and the current SpO₂ level is low (e.g., below 75%), then the usual algorithms and calibration tables may be used because the remaining 730 nm-900 nm emitter pair remains suitable for providing more accurate measurements at lower arterial oxygen saturation levels. If the current SpO₂ level is high (e.g., above 85%), then the 730 nm-900 nm emitter pair may still provide useable measurements. Accordingly, a recovery or limp mode of operation may be used that operates the 730 nm-900 nm pair by using, for example, calibration curves suitable for deriving measurements over a wider range of arterial oxygen saturation levels, including lower and transition region saturation levels. Likewise, if the 730 nm emitter becomes inoperable and the current SpO₂ level is high, then the 660 nm-900 nm emitter pair may continue providing more accurate measurements. If the SpO₂ level is low, then the 660 nm-900 nm emitter pair may still provide clinically useful measurement, for example, by using calibration curves suitable for deriving SpO₂ measurements at lower and transition region saturation levels. In this way, useful SpO₂ measurements may be provided in the event of failures of the 660 nm or the 730 nm emitters. Because 990 nm emitter may be used to derive all SpO₂ calculations, a failure of the 990 nm may result in a suspend code that suspends SpO₂ measurements and that alerts the user or the clinician to replace the sensor. Alternatively, the 660 nm and 730 nm emitters may be used to measure SpO₂ if the accuracy is suitable. Other alerts may be provided that inform the user or the clinician of the status of all of the emitters and the accuracy of current measurements, as described in more detail below.

It should be noted that spectrophotometric systems that incorporate multiple emitters may benefit from the techniques disclosed herein, such as systems for hemometry and aquametry. Indeed, the techniques disclosed herein may be suitable for use in a variety of devices that include two or more light emitters. For example, hemometers, aquameters, and so forth, may also incorporate the techniques disclosed herein to provide for a recovery or limp mode of operation suitable for using the light emitters that remain operational.

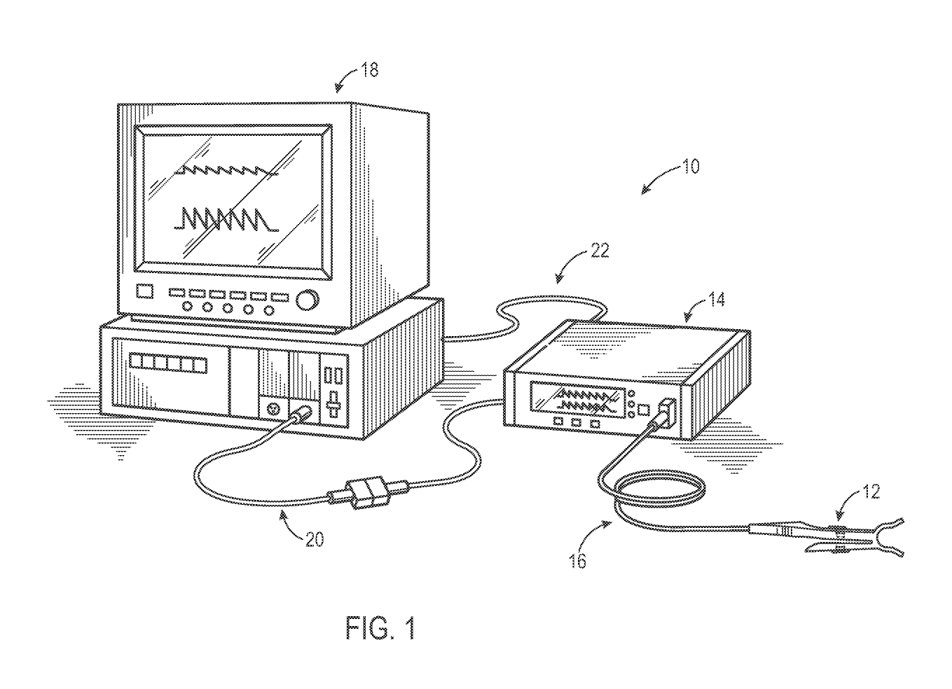

FIG. 1 depicts a spectrophotometric system 10, such as a pulse oximeter, hemometer and/or aquameter. The sensor 12 may be coupled to the monitor 14 via sensor cable 16. The spectrophotometric system 10 may be any suitable pulse oximeter, hemometer and/or aquameter, such as those available from Nellcor Puritan Bennett LLC. Furthermore, to upgrade conventional operation provided by the monitor 14 to provide additional functions, monitor 14 may be coupled to a multi-parameter patient monitor 18 via a cable 20 connected to a sensor input port or via a cable 22 connected to a digital communication port, for example.

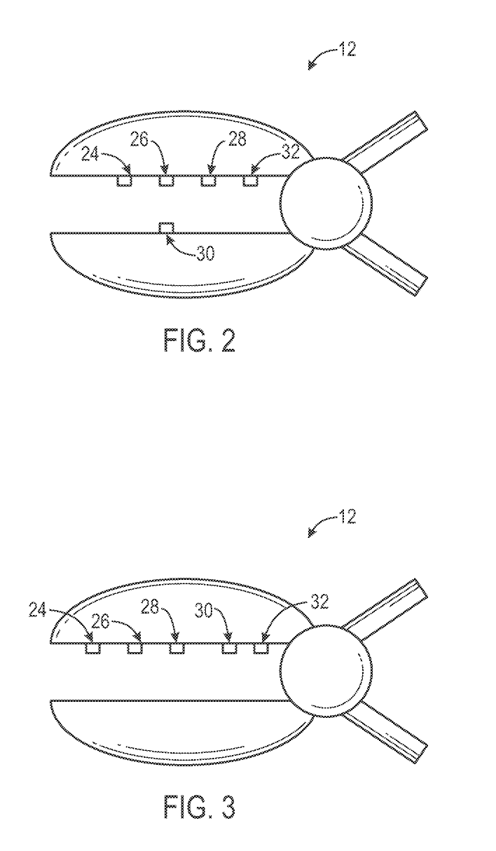

In one embodiment, the sensor may include three LEDs emitting at different wavelength ranges, as depicted in more detail below with respect to FIGS. 2 and 3. For example, in a pulse oximetry embodiment, the three wavelength ranges may include a red wavelength at approximately 620-700 nm; a far red wavelength at approximately 690-770 nm; and an infrared wavelength at approximately 860-940 nm. In a specific embodiment, light within each one of the three wavelength ranges may be respectively emitted by a 660 nm emitter, a 730 nm emitter, and a 900 nm emitter. For example, by analyzing the light emitted by the 660 nm emitter and by the 900 nm emitter, a more accurate measurement may be obtained in the high SpO₂ range (e.g., greater than 85%). Likewise, a more accurate measurement may be derived in the low SpO₂ range (e.g., lower than 75%) by analyzing the light emitted by the 730 nm and by the 900 nm emitter. It is to be understood that, in other embodiments, more emitters may be used and/or different wavelengths may be emitted. For example, in a hemometry embodiment, four emitted wavelengths may be used. The emitted wavelength ranges may include a blue wavelength at approximately 430-510 nm, a green wavelength at approximately 480-530 nm, a far red wavelength at approximately 690-770 nm, and an infrared wavelength at approximately 860-940 nm. By using the techniques disclosed herein, the failure of one (or more) of the sensor 12 emitters may be overcome by deriving useful measurements from the emitters that remain operational.

Turning to FIG. 2, the figure illustrates a transmission type sensor 12 wherein light from an LED emitter 24, light from an LED emitter 26, and light from an LED emitter 28 pass through tissue to reach a detector 30 on the other side of the tissue. FIG. 3 depicts a reflectance type sensor 12 wherein the LED emitter 24, the LED emitter 26, the LED emitter 28, and the detector 30 are all positioned on the same side of the sensor 12 so that the emitted light is reflected through the vascularized tissue underneath the emitters back into the detector 30. As mentioned above, the sensor 12 may be a pulse oximetry sensor. Accordingly, the emitters 24, 26, and 28 may emit light at approximately 660 nm, 730 nm, and 900 nm respectively. In a hemometry example, the emitters 24, 26, and 28 may emit light at approximately 475 nm, 510 nm, and 730 nm respectively. A fourth light emitter 32 may be also used for hemometry measurements. This fourth light emitter 32 may emit light at approximately 990 nm. It should be noted that more or less emitters may be used, depending on the sensing capabilities included in the sensor 12. It should also be noted that the spacing of the emitters and the detectors of FIGS. 2 and 3 are for illustrative purposes and not to scale. Indeed, the same light path length for all emitter-detector pairs is typical, and accordingly, the LEDs may be positioned in close proximity to each other.

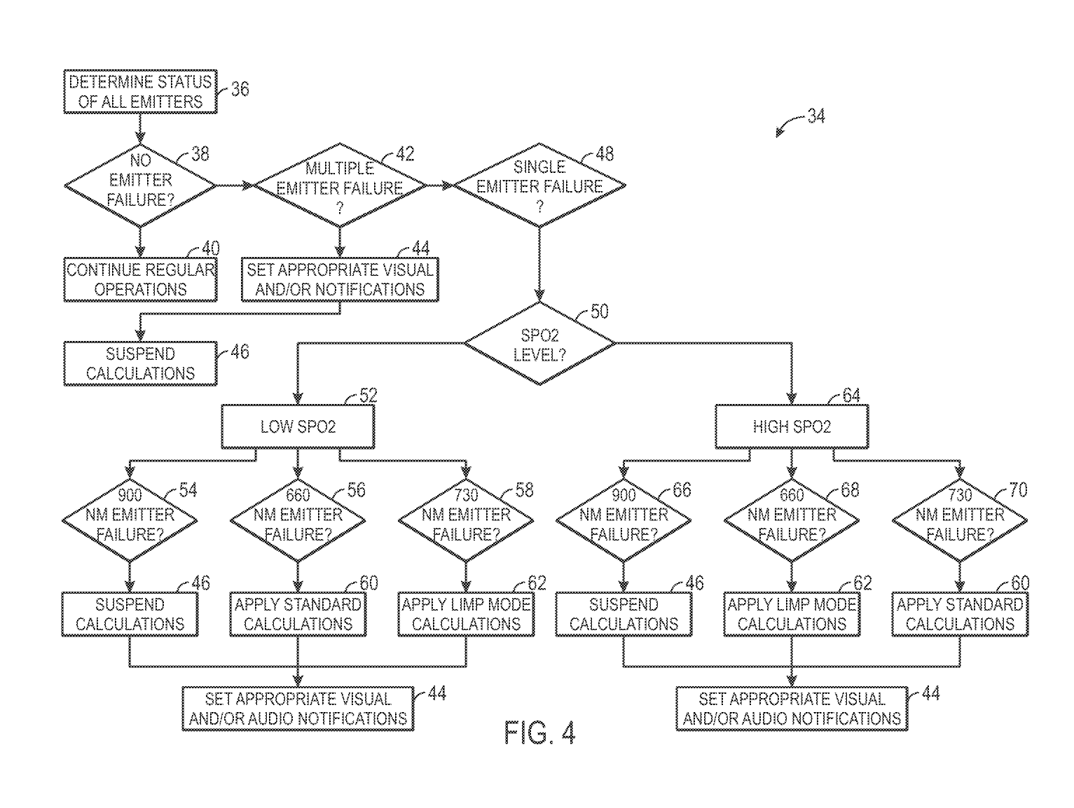

In one example, all of the emitters 24, 26, 28, and 32 may be alternatively used to illuminate a patient tissue. The emitted light may then be detected and used to calculate measurements, such as SpO₂ measurements. For example, calibration coefficient tables may be used with suitable algorithms to convert the measured red and infrared signals (or far red and infrared signals) into the SpO₂ measurements. As mentioned above, the red and infrared emitters (i.e., emitters 24 and 28) may be used to more accurately measure SpO₂ when the arterial oxygen saturation is high (e.g., greater than 85%). The far red and infrared emitters (i.e., emitters 26 and 28) may be used to more accurately measure SpO₂ when the arterial oxygen saturation is low (e.g., below 75%). Further, the emitters 24, 26, and 28 may be used to more accurately measure SpO₂ when the arterial oxygen saturation is in a transition region, e.g., the region between the low and high oxygen saturation. In cases were one or more of the emitters 24, 26, 28 were to become dysfunctional or otherwise inoperable, a certain recovery or limp mode logic may be used to continue deriving useful measurements, as described in more detail below with respect to FIG. 4.

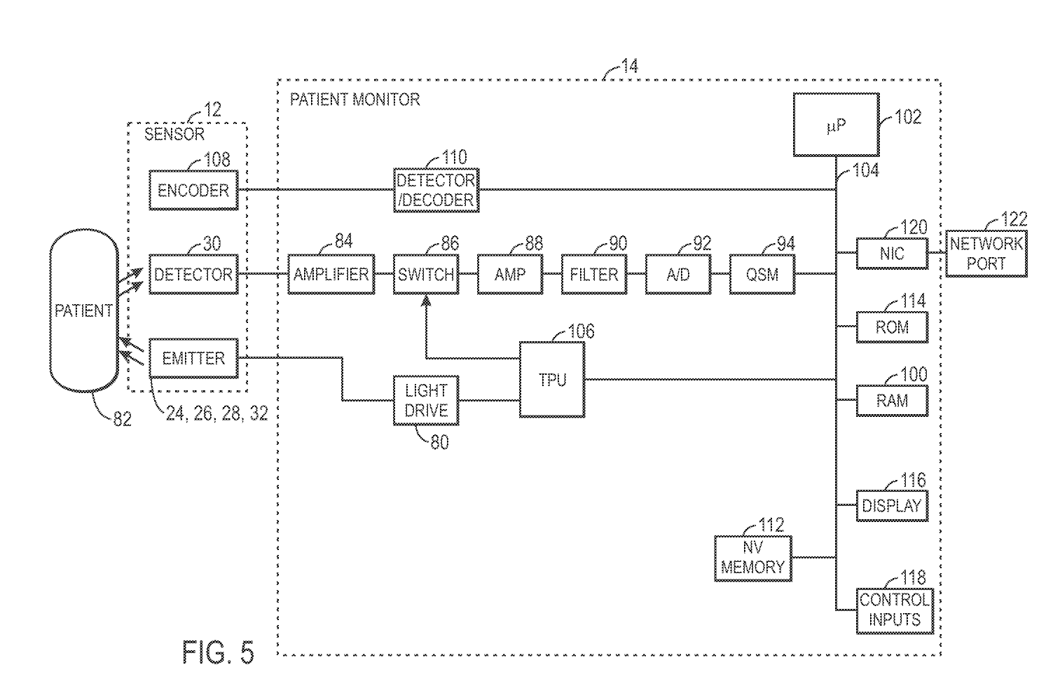

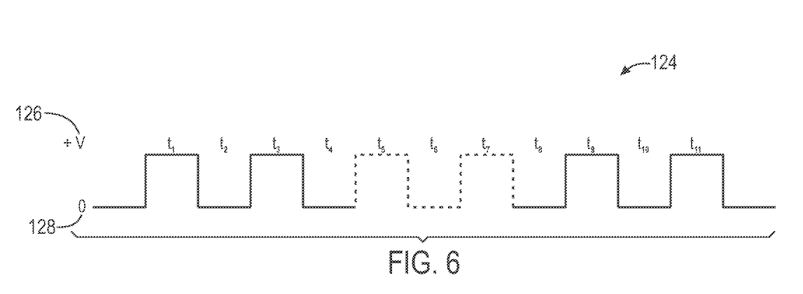

FIG. 4 is a flow diagram depicting one embodiment of a logic 34 that may used to derive SpO₂ measurements, even in certain circumstances where one or more emitters 24, 26, 28, and 32 of the sensor 12 may become inoperable. The logic 34 may first determine the operational status of all emitters (block 36). The operational status of the emitters may be determined through a variety of techniques. For example, the sensor may include circuitry as described in more detail below with respect to FIG. 5, suitable for detecting which emitters are currently operating within desired parameters. In another example, the signals from the detector 30 may be analyzed as described in more detail below with respect to FIG. 6, to determine the operational status of the emitters 24, 26, 28 and 32.

If the logic 34 determines that there are no emitter failures (decision 38), then the logic 34 may continue with regular operations of the sensor 12 (block 40). If it is determined that there are multiple emitter failures (decision 42), then the logic 34 may set appropriate visual and/or audio notifications (block 44). The visual notifications may include, for example, a numeric code and/or textual messages indicative of the current status of the sensor 12, which may then be displayed for viewing by a user and/or clinician. For example, the textual messages may list the emitters that are no longer operative, and may indicate the need to replace the sensor 12. The visual notifications may also list any measurements that are no longer obtainable. If only a single emitter is left operational, the logic 34 may then suspend SpO₂ calculations (block 46). It is to be understood that other calculations may still be performed with a single emitter, such as pulse rate calculations. Additionally, audible notifications may be provided, such as beeps or other aural tones, suitable for attracting attention to the display of the visual messages. Such audible notifications may further aid in informing the user and/or clinician of the operational status of the sensor 12.

If it is determined that only a single emitter has failed (decision 48), the logic 34 may then determine the SpO₂ level (decision 50) of the patient undergoing observation. For example, previously valid measurements may be logged at certain time periods (e.g., every second) and the most recent valid measurement may then be used to determine the SpO₂ level. If it is determined that the SpO₂ measurement is currently at a low SpO₂ level (block 52), then the logic 34 may determine which one of the emitters has failed. If it is determined that the 900 nm emitter has failed (decision 54), then the logic 34 may suspend calculations. The 900 nm emitter is used in all SpO₂ ranges, so the failure of this emitter may prevent useful SpO₂ calculations. The logic 34 may then set the appropriate visual and/or audio notifications (block 44) to alert of the suspension of SpO₂ calculations and to advise for the replacement of the sensor 12, as described above. Alternatively, the logic 34 may continue calculating SpO₂ using the remaining emitters, providing, if necessary, a suitable warning of reduced accuracy or reliability of the measurements.

If it is determined that the 660 nm emitter has failed (decision 56), then the logic 34 may apply standard calculations and algorithms (block 60), for example, by using the 730 nm-900 nm emitter pair. Indeed, the 730 nm-900 nm pair may be used with calibration tables optimized for lower SpO₂ ranges in order to derive SpO₂ measurements having improved accuracy at the lower ranges. The logic 34 may then set the appropriate visual and/or audio notifications (block 44). The notifications may include warnings that the 660 mm emitter has failed, as well as text detailing that while the current SpO₂ measurements may be accurate, less accuracy may be derived at higher SpO₂ levels.

If it is determined that the 730 nm emitter has failed (decision 58), then the logic 34 may apply a limp mode set of calculations (block 62), for example, by using the 660 nm-900 nm emitter pair. In one embodiment, broader calibration coefficient tables may be used suitable for deriving useful values over a wider range of arterial oxygen saturation levels. Indeed, the calibration tables may be used with the 660 nm-900 nm emitter pair over all ranges of arterial oxygen saturation, but may be less accurate at lower SpO2 ranges. The logic 34 may then set the appropriate visual and/or audio notifications (block 44). The notifications may include warnings that the 730 nm emitter has failed, as well as text detailing that the current SpO₂ measurements may not be as accurate.

If it is determined that the SpO₂ measurement is currently at a high SpO₂ level (block 64), then the logic 34 may determine which one of the emitters has failed. For example, if it is determined that the 900 nm emitter has failed (decision 66), then the logic 34 may suspend SpO₂ calculations. As mentioned above, the 990 nm emitter may be used in all SpO₂ calculations. Accordingly, the logic 34 may have to suspend the SpO₂ calculations (block 46). The logic 34 may then proceed to set the appropriate visual and/or audio notifications (block 44) informing of the stoppage of the SpO₂ measurements and advising on the replacement of the sensor 12. Alternatively, logic 34 may continue calculating SpO₂ using the remaining emitters, providing, if necessary, a warning of reduced measurement accuracy and reliability.

If it is determined that the 660 nm emitter has failed (decision 68), then the logic 34 may apply a limp mode set of calculations (block 62), for example, by using the 730 nm-900 nm emitter pair. In one embodiment, broader calibration coefficient tables may be used suitable for deriving useful values over a wider range of arterial oxygen saturation levels, including the higher arterial oxygen saturation levels. Indeed, the calibration tables may be used with the 730 nm-900 nm emitter pair over all ranges of arterial oxygen saturation, but may be less accurate at higher SpO₂ ranges. The logic 34 may then set the appropriate visual and/or audio notifications (block 44). The notifications may include warnings that the 730 nm emitter has failed, as well as text detailing that the current SpO₂ measurements may not be as accurate.

If it is determined that the 730 nm emitter has failed (decision 70), then the logic 34 may apply standard calculations and algorithms (block 60), for example, by using the 660 nm-900 nm emitter pair. As mentioned above, the 660 nm-900 nm pair may be used with calibration tables optimized for higher SpO₂ ranges so as to derive SpO₂ measurements having improved accuracy at the lower ranges. The logic 34 may then set the appropriate visual and/or audio notifications (block 44). The notifications may include warnings that the 730 nm emitter has failed, as well as text detailing that while the current SpO₂ measurements may be very accurate, less accuracy may be derived at lower SpO₂ levels. By selectively using, for example, calibration tables suitable for deriving useful SpO₂ values at broader ranges, the logic 34 may enable useful measurements even in situations when one of the emitters of the sensor 12 is not fully operational.

FIG. 5 depicts a block diagram of an example pulse oximeter that may be configured to implement certain techniques described above. In one example, a light drive circuitry 80 may drive the emitters 24, 26, 28, and 32. Light from emitters 24, 26, 28, or 32 passes into a patient's blood perfused tissue 82 and is detected by detector 30. The detected light may be converted into detector 30 signals. The detector 30 signals may then be passed through an amplifier 84, a switch 86, a post-switch amplifier 88, a low band filter 90, and an analog-to-digital converter 92. The digital data may then be stored in a queued serial module (QSM) 94 for later downloading to RAM 100 as QSM 94 fills up.

In certain embodiments, based at least in part upon the value of the received signals corresponding to the light detected by detector 30 as explained in further detail with respect to FIG. 6 below, a microprocessor 102 may obtain the operation status of the emitters 24, 26, 28, and 32. That is, the microprocessor 102 may determine if any of the emitters 24, 26, 28, and 32 has become inoperable or unreliable by analyzing the detected light. In other examples, the microprocessor 102 may receive certain signals from the sensor 12 indicative of emitter malfunction. For example, if the emitters are LED emitters, certain circuitry in the sensor 12 (or in the monitor 14) may measure resistance for each LED and detect a short circuit or other resistance change in the LED circuitry. Any suitable detection circuitry may be used, such as a Wheatstone bridge, suitable for measuring the resistance values of the emitters 24, 26, 28, and 32. Signals representative of emitter malfunctions may then be sent, for example, to the monitor 14 and used in determining the set of operable emitters. The microprocessor 102 may then employ the logic 34, to calculate SpO₂ values, as described above with respect to FIG. 4. In this way, the microprocessor 102 may use a working subset of the emitters 24, 26, 28, and 32 to derive various measurements, such as arterial oxygen saturation measurements.

In one embodiment, also connected to a bus 104 may be a time processing unit (TPU) 106 that may provide timing control signals to light drive circuitry 80. The sensor 12 may also use an encoder 108 for encryption coding that prevents a disposable part of the sensor 12 from being recognized by a detector/decoder 110 that is not able to decode the encryption. In some embodiments, the encoder 108 and/or the detector/decoder 110 may not be present. Additionally or alternatively, the processor 112 may encode and/or decode processed sensor data before transmission of the data to the patient monitor 14.

Nonvolatile memory 112 may store caregiver preferences, patient information, or various parameters such as the calibration tables discussed above with respect to FIG. 4, which may be used during the limp mode of operation of the sensor 12. Software for performing the configuration of the monitor 14 and for carrying out the techniques described herein may also be stored on the nonvolatile memory 112, or may be stored on the ROM 114. The visual notifications of the operational status of the sensor 12, as well as other may be displayed by display 116 and manipulated through control inputs 118. A network interface card (NIC) 120 may be connected to a network port 122 and used to deliver, for example, the operational status of the sensor 12, any alerts or notifications, and physiologic measurements.

FIG. 6 depicts an example of the use of a detected light signal 124 to determine the operational status of an emitter. More specifically, the microprocessor 102 may use a detected light signal, such as the signal 124, to detect if an emitter, such as the emitter 24, 26, 28, and 32, is not emitting light in a manner suitable for use in spectrophotometric operations. The first on signal (+V) 126 at time period t₁ denotes that the light was detected. The first off signal (0 V) 128 at time period t₂ denotes that no light was detected. It is to be understood that due to, for example, a slight leakage of ambient light, the off or “dark time” signal 128 may be slightly larger than 0 V. The next on signal (+V) 126 at time t₃ denotes that light was detected. The next off signal (0 V) 128 at time t₄ denotes that no light was detected. Up to and including time t₄, the depicted on/off pattern of detected light may be in accordance to normal multiplexing operations of the emitter. However, the expected signals 126 at time t₅ and t₇ are not received. Should no further signal 126 be received, the microprocessor 102 may infer that the emitter is no longer operational or is otherwise unsuitable for use in deriving measurements.

Should the signal 126 resume at times t₉ and t₁₁, as illustrated, the microprocessor 102 may infer that the light was either temporarily not detected (e.g., the sensor 12 was temporarily moved out of position from the patient tissue) or that the emitter is functioning erratically. In one embodiment, the microprocessor 102 may keep track of the number of occurrences of missing signals 126 and set the status of the emitter as “inoperable” based on a certain threshold number of missing signals 126. For example, if the number of missing signals 126 over a time period (e.g., five milliseconds) exceeds 10 missing signals 126, then the microprocessor 102 may infer that the emitter is not operating within desired parameters. Indeed, by detecting missing values of expected light signals, the microprocessor 102 may derive the functional status of any one of the emitters 24, 26, 28, and 32. Accordingly, the microprocessor 102 may use a logic, such as the logic 34 described above with respect to FIG. 4, to query the status of all emitters and to derive useful measurements based on the emitters that remain operable.

What is claimed is:

1. A monitor comprising:
a processing circuit configured to drive a sensor having three light emitters of three different wavelengths and to derive a physiological measurement using signals received from at least two of the light emitters, where the processing circuit is further configured to determine whether any of the three emitters has failed, and if one of the emitters has failed, to continue to derive the physiological measurement from the other two emitters and to provide an indication related to the failed emitter.

2. The monitor, as set forth in claim 1, wherein, upon determining that the one emitter has failed, the processing circuit is configured to determine a level of the physiological measurement and to apply standard calculations or alternative calculations to derive the physiological measurement depending upon which of the three emitters failed.

3. The monitor, as set forth in claim 1, wherein the physiological measurement comprises a blood oxygenation level, and wherein the three wavelengths comprise a first red wavelength from a first emitter substantially optimized for high blood oxygenation levels, a second red wavelength from a second emitter substantially optimized for low blood oxygenation levels, and an infrared wavelength from a third emitter.

4. The monitor, as set forth in claim 3, wherein if the processing circuit determines that the first emitter has failed and determines that blood oxygenation level is a low blood oxygenation level, the processing circuit continues to drive the second emitter and the third emitter, determine the blood oxygenation level based on an algorithm substantially optimized for determining a low blood oxygenation level, and causes an indication that one of the three emitters has failed; and wherein if the processing circuit determines that the first emitter has failed and determines that blood oxygenation level is a high blood oxygenation level, the processing circuit continues to drive the second emitter and the third emitter, determine the blood oxygenation level based on an alternative algorithm, and causes an indication that one of the three emitters has failed and that the blood oxygenation calculations may not be accurate.

5. The monitor, as set forth in claim 3, wherein if the processing circuit determines that the second emitter has failed and determines that blood oxygenation level is a high blood oxygenation level, the processing circuit continues to drive the first emitter and the third emitter, determine the blood oxygenation level based on an algorithm substantially optimized for determining a high blood oxygenation level, and causes an indication that one of the three emitters has failed; and wherein if the processing circuit determines that the second emitter has failed and determines that blood oxygenation level is a low blood oxygenation level, the processing circuit continues to drive the first emitter and the third emitter, determine the blood oxygenation level based on an alternative algorithm, and causes an indication that one of the three emitters has failed and that the blood oxygenation calculations may not be accurate.

6. The monitor, as set forth in claim 3, wherein if the processing circuit determines that the third emitter has failed, the processing circuit suspends derivation of blood oxygenation level and causes an indication that the sensor should be replaced.

7. A system for determining a physiological measurement, the system comprising:
a sensor having at least three light emitters, each of the emitters being configured to emit light at a different wavelength;
a monitor configured to drive the at least three emitters of the sensor and to derive a physiological measurement using signals received from at least two of the emitters, where the monitor is further configured to determine whether any of the at least three emitters has failed, and if one of the emitters has failed, to continue to derive the physiological measurement from at least two of the operable emitters and to provide an indication related to the failed emitter.

8. The system, as set forth in claim 7, wherein, upon determining that the one emitter has failed, the monitor is configured to determine a level of the physiological measurement and to apply standard calculations or alternative calculations to derive the physiological measurement depending upon which of the at least three emitters failed.

9. The system, as set forth in claim 7, wherein the physiological measurement comprises a blood oxygenation level, and wherein the at least three emitters comprise a first emitter having a first red wavelength substantially optimized for high blood oxygenation levels, a second emitter having a second red wavelength substantially optimized for low blood oxygenation levels, and a third emitter having an infrared wavelength.

10. The system, as set forth in claim 9, wherein if the monitor determines that the first emitter has failed and determines that blood oxygenation level is a low blood oxygenation level, the monitor continues to drive the second emitter and the third emitter, determine the blood oxygenation level based on an algorithm substantially optimized for determining a low blood oxygenation level, and causes an indication that one of the three emitters has failed; and wherein if the monitor determines that the first emitter has failed and determines that blood oxygenation level is a high blood oxygenation level, the monitor continues to drive the second emitter and the third emitter, determine the blood oxygenation level based on an alternative algorithm, and causes an indication that one of the three emitters has failed and that the blood oxygenation calculations may not be accurate.

11. The system, as set forth in claim 9, wherein if the monitor determines that the second emitter has failed and determines that blood oxygenation level is a high blood oxygenation level, the monitor continues to drive the first emitter and the third emitter, determine the blood oxygenation level based on an algorithm substantially optimized for determining a high blood oxygenation level, and causes an indication that one of the three emitters has failed; and wherein if the monitor determines that the second emitter has failed and determines that blood oxygenation level is a low blood oxygenation level, the monitor continues to drive the first emitter and the third emitter, determine the blood oxygenation level based on an alternative algorithm, and causes an indication that one of the three emitters has failed and that the blood oxygenation calculations may not be accurate.

12. The system, as set forth in claim 9, wherein if the monitor determines that the third emitter has failed, the monitor suspends derivation of blood oxygenation level and causes an indication that the sensor should be replaced.

13. A method for determining a physiological measurement, the method comprising:
driving a sensor having three light emitters of three different wavelengths;
deriving a physiological measurement using signals received from at least two of the light emitters;
determining whether any of the three emitters has failed;
if one of the emitters has failed, continuing to derive the physiological measurement from the other two emitters and providing an indication related to the failed emitter.

14. The method, as set forth in claim 13, wherein, upon determining that the one emitter has failed, determining a level of the physiological measurement and applying standard calculations or alternative calculations to derive the physiological measurement depending upon which of the three emitters failed.

15. The method, as set forth in claim 13, wherein the physiological measurement comprises a blood oxygenation level, and wherein the three wavelengths comprise a first red wavelength from a first emitter substantially optimized for high blood oxygenation levels, a second red wavelength from a second emitter substantially optimized for low blood oxygenation levels, and an infrared wavelength from a third emitter.

16. The method, as set forth in claim 15, wherein:
if the first emitter has failed and the blood oxygenation level is a low blood oxygenation level, continuing to drive the second emitter and the third emitter, determining the blood oxygenation level based on an algorithm substantially optimized for determining a low blood oxygenation level, and causing an indication that one of the three emitters has failed; and
if the first emitter has failed and the blood oxygenation level is a high blood oxygenation level, the continuing to drive the second emitter and the third emitter, determining the blood oxygenation level based on an alternative algorithm, and causing an indication that one of the three emitters has failed and that the blood oxygenation calculations may not be accurate.

17. The method, as set forth in claim 15, wherein:
if the second emitter has failed and the blood oxygenation level is a high blood oxygenation level, continuing to drive the first emitter and the third emitter, determining the blood oxygenation level based on an algorithm substantially optimized for determining a high blood oxygenation level, and causing an indication that one of the three emitters has failed; and
if the second emitter has failed and the blood oxygenation level is a low blood oxygenation level, continuing to drive the first emitter and the third emitter, determining the blood oxygenation level based on an alternative algorithm, and causing an indication that one of the three emitters has failed and that the blood oxygenation calculations may not be accurate.

18. The method, as set forth in claim 15, wherein if the processing the third emitter has failed, suspending derivation of blood oxygenation level and causing an indication that the sensor should be replaced.
